(19) United States
(12) Patent Application Publication
(10) Pub. No.: US 2011/0021691 A1
(43) Pub. Date: Jan. 27, 2011

(54) Antistatic ionic compound, oligomer thereof, copolymer thereof, and pressure-sensitive adhesive composition

(76) Inventor: Hou-Jen Chiang, Taoyuan County (TW)

Correspondence Address: NORTH AMERICA INTELLECTUAL PROPERTY CORPORATION, P.O. BOX 506, MERRIFIELD, VA, 22116, US

(21) Appl. No.: 12/826,649

(22) Filed: Jun. 29, 2010

(30) Foreign Application Priority Data
Jul. 22, 2009 (TW) 098124714

(51) Int. Cl. C08L 39/00; C07C 69/533; C08F 26/02; C08L 35/02

(52) U.S. Cl. 524/521; 560/222; 526/312; 524/502

(57) ABSTRACT

A pressure-sensitive composition contains an ionic compound, or an oligomer or co-polymer of the ionic compound which is composed of a ((meth) acryloyloxy) alkyl ammonium cation and (CF₃SO₂)₂N⁻ anion, or a combination thereof, to obtain a pressure-sensitive adhesive sheet having excellent antistatic properties without adhesion deterioration.

### BACKGROUND OF THE INVENTION

1. Field of the Invention

The present invention relates to an antistatic pressure-sensitive adhesive composition, and particularly to an antistatic component and an acrylic pressure-sensitive adhesive composition containing the antistatic component, which has both excellent adhesion and antistatic properties.

2. Description of the Prior Art

Pressure-sensitive adhesives (PSAs) are adhesives that can bind two types of materials together upon the application of a contact pressure. They are widely and conveniently used and have been utilized in display devices to serve as, for example, adhesive layers for polarizing sheets in liquid crystal displays.

Conventional pressure-sensitive adhesives are composed of organic polymeric materials and typically have highly electric insulation properties, such that statistic charges tend to occur upon rubbing or peeling. Accordingly, when a releasing film for protecting a pressure-sensitive adhesive sheet is removed from the pressure-sensitive adhesive sheet for binding the polarizing sheet and an LCD panel, static charges occur to the pressure-sensitive adhesive sheet due to friction. It may result in pollution to optical members due to dust attachment or result in electrostatic breakage to peripheral circuit members, which, in turn, results in abnormal display due to liquid crystal orientation disorder. For avoiding the aforesaid defects, an antistatic treatment is needed for the pressure-sensitive adhesive layer.

It is common and effective to improve electric conductivity of material for solving problems caused by static charge accumulation. The improvement of the conductivity of material by increasing moisture adsorption is frequently adopted to control the static charge accumulation. Increase of moisture adsorption can be generally accomplished by increasing ambient moisture or using moisture adsorption antistatic agent. Most antistatic agents are used for dissipating accumulated static charges.

Conventional chemical antistatic species may include organic materials, such as, organic amines, amides, polyoxyethylene derivatives, polyols and the like, inorganic materials, such as metal powder, carbon powder, inorganic salts and the like, and even neutral or ionic surfactants. When the pressure-sensitive adhesive is added with metal powder or carbon powder, its optical properties will be influenced due to poor transparency. When the pressure-sensitive adhesive is added with a polyoxyethylene derivative, its adhesion performance will be influenced due to the migration of such surfactant to the surface of the pressure-sensitive adhesive. The alkali metal cation, especially lithium cation, is also commonly used as an antistatic agent due to good moisture adsorption. Room temperature molten salt, such as anionic liquid, can also serve as an antistatic agent. However, it should pay attention to the compatibility of the antistatic agent with the polymer in the polymeric composition; otherwise inappropriate antistatic performance will occur or the final products or articles may have significantly improper physical properties or appearance.

Therefore, there is still a need for a novel antistatic agent and a pressure-sensitive adhesive containing such antistatic agent, both having good compatibility and resulting in a pressure-sensitive adhesive sheet having excellent antistatic properties without influencing the adhesion performance.

### SUMMARY OF THE INVENTION

One objective of the present invention is to provide an ionic compound which may function as an antistatic agent and a pressure-sensitive adhesive composition containing such ionic compound as an antistatic component. Such ionic compound is compatible with the pressure-sensitive adhesive component, resulting in the pressure-sensitive adhesive composition having excellent adhesion reliability due to its excellent antistatic properties without deteriorating adhesion performance.

The ionic compound according to the present invention is represented by the following chemical formula (I):

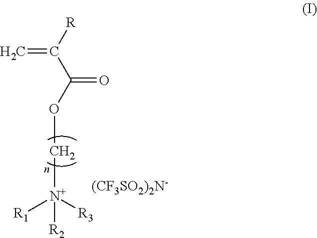

wherein, R is a hydrogen atom or a methyl group; R₁, R₂, and R₃ are each independently an alkyl group having 1 to 6 carbon atoms; and n is an integer of 1 to 16.

In another aspect of the present invention, an oligomer is provided, which comprises a product produced from oligomerization of the aforesaid ionic compound.

In still another aspect of the present invention, a copolymer is provided, which comprises a product produced from copolymerization of comonomers including the aforesaid ionic compound and at least an acryloyl-reactive comonomer.

In further another aspect of the present invention, a pressure-sensitive adhesive composition is provided, which comprises a first component selected from the group consisting of the aforesaid ionic compound, the aforesaid oligomer, the aforesaid copolymer and a combination thereof and a second component comprising a pressure-sensitive adhesive polymer.

The pressure-sensitive adhesive composition of the present invention contains a novel ionic compound, an oligomer or a copolymer thereof. The novel ionic compound is essentially composed of ((meth)acryloyloxy)alkyl ammonium cation and (CF₃SO₂)₂N⁻ anion, allowing the obtained pressure-sensitive adhesive sheet to have excellent antistatic properties without adhesion deterioration. Accordingly, the pressure-sensitive adhesive sheet can be suitably used in plastic products which tend to produce static charges and articles for which static charges are not desired, for example, electronic products, such as polarizing sheets.

These and other objectives of the present invention will no doubt become obvious to those of ordinary skill in the art after reading the following detailed description of the preferred embodiment that is illustrated in the various figures and drawings.

### DETAILED DESCRIPTION

The ionic compound according to the present invention which may serve as an antistatic component may be represented by the following chemical formula (I):

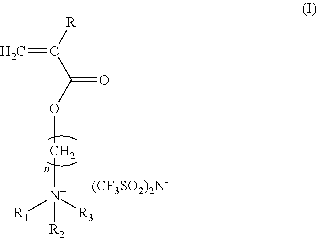

wherein, R is a hydrogen atom or a methyl group, and R₁, R₂ and R₃ are each independently an alkyl group. In consideration of stereo-hindrance and feasibility of reaction, it is preferred that R₁, R₂ and R₃ are each independently an alkyl group having 1 to 6, and more preferably 1 to 4 carbon atoms. “n” is an integer of 1 to 16, preferably 1 to 8, and more preferably 1 to 6. It is more preferred that the carbon chain, —(CH)ₙ—, is longer than that of R₁, R₂ and R₃. When n is 2 or more, the effect is more improved.

Such ionic compound is essentially composed of ((meth)acryloyloxy)alkyl ammonium cation and (CF₃SO₂)₂N⁻ anion. “(meth)acryl” is referred to as “acryl” or “methacryl”. Because the cation portion of the ionic compound has a (meth)acryl moiety, the ionic compound is well compatible with an acrylic pressure-sensitive adhesive component.

In another aspect of the present invention, the oligomer formed from the aforesaid ionic compound represented by the chemical formula (I) can also serve as a good antistatic component. Such oligomer is obtained by polymerization of the aforesaid ionic compound functioning as the monomer. The polymerization site is the double bond on the acrylic group. The type of polymerization is not limited to a particular one. Solution polymerization, bulk polymerization, suspension polymerization, emulsion polymerization are photo-polymerization are preferred, and solution polymerization is more preferred. The polymerization may be initiated by an initiator. The initiators usable for the polymerization may include azo type initiators, such as azobisisobutyronitrile and azobiscyclohexanenitrile, and peroxides, such as benzoyl peroxide and acetyl peroxide. These initiators may be used in combination. The polymerization may be carried out using the initiator at 50 to 100° C.

In another aspect of the present invention, the polymerization is carried out as the above, but the monomer is not limited to the ionic compound represented by the chemical formula (I) and can be others. The ionic compound represented by the chemical formula (I) may be used as a comonomer with an acryloyl-reactive comonomer to perform a co-polymerization to obtain a copolymer. The acryloyl-reactive comonomers may include for example (meth)acrylic monomer, such as methyl (meth)acrylate, ethyl (meth)acrylate, n-butyl (meth)acrylate and 2-ethylhexyl (meth)acrylate; and vinyl monomer, such as vinyl acetate, styrene and vinyl pyrrolidone. They can be used in combination. The comonomers are mixed and copolymerized, resulting in a copolymer also having antistatic properties and being suitably used in the pressure-sensitive adhesive composition.

The pressure-sensitive adhesive composition according to the present invention includes a pressure-sensitive adhesive polymer as a component and the ionic compound represented by the chemical formula (I), an oligomer thereof, a copolymer thereof or a combination thereof as another component. The amounts of these two components are not particularly limited. In consideration of the adhesion and the antistatic properties of the obtained pressure-sensitive adhesive composition, the ionic compound represented by the chemical formula (I) (as calculated by the ionic compound when the oligomer or copolymer are utilized) is preferably used in an amount of about 0.01 to 20% by weight, based on a total weight of these two components as 100% by weight. The oligomer suitably utilized in the pressure-sensitive adhesive composition may have a molecular weight of for example 1,000 to 100,000, and preferably 5,000 to 50,000, or the number of the ionic compound as the repeating unit may be about 10 to about 120. In consideration of the hydrophilicity of the monomer, n of the ionic compound represented by the chemical formula (I) is preferably 1 to 8, and more preferably 1 to 6. When n is 2 or more, the antistatic effect is more improved.

The pressure-sensitive adhesive composition according to the present invention may further include a solvent or solvents. The solvents may include for example a typical organic solvent, such as acetone, toluene, ethyl acetate and the like, in which the acrylate may be dissolved. The solvent or solvents may be used in an amount of about 30 to 85% by weight, and preferably about 50 to 80% by weight, of the pressure-sensitive adhesive composition. The pressure-sensitive adhesive composition of the present invention may be formed by mixing a conventional pressure-sensitive adhesive polymer or the solution thereof with the ionic compound represented by the chemical formula (I) or the oligomer or the copolymer of the ionic compound represented by the chemical formula (I).

The pressure-sensitive adhesive polymer is preferably a (meth)acryl-based polymer, which is well compatible with the ionic compound of the present invention and the resulting adhesive can maintain sufficient adhesion performance. Such (meth)acryl-based polymer preferably contains one or more alkyl (meth)acrylates having the alkyl group having 1 to 14 carbon atoms as a main component. Alkyl (meth)acrylates having the alkyl group having 1 to 14 carbon atoms may include for example methyl (meth)acrylate, ethyl (meth)acrylate, n-butyl (meth)acrylate, s-butyl (meth)acrylate, t-butyl (meth)acrylate, iso-butyl (meth)acrylate, hexyl (meth)acrylate, 2-ethylhexyl (meth)acrylate, n-octyl (meth)acrylate, iso-octyl (meth)acrylate, n-nonyl (meth)acrylate, iso-nonyl (meth)acrylate, n-decyl (meth)acrylate, iso-decyl (meth)acrylate, n-dodecyl (meth)acrylate, n-tridecyl (meth)acrylate, n-tetradecyl (meth)acrylate and the like.

Among these, n-butyl (meth)acrylate, s-butyl (meth)acrylate, t-butyl (meth)acrylate, iso-butyl (meth)acrylate, hexyl (meth)acrylate, 2-ethylhexyl (meth)acrylate, n-octyl (meth)acrylate, iso-octyl (meth)acrylate, n-nonyl (meth)acrylate, iso-nonyl (meth)acrylate, n-decyl (meth)acrylate, iso-decyl (meth)acrylate and the like are preferably used in the present invention.

The pressure-sensitive adhesive polymer may be obtained through polymerization of monomers based on one or more types of alky (meth)acrylate having the alkyl group having 1 to 14 carbon atoms, which may be preferably contented in a total amount of 50-99.9% by weight, more preferably 60-99% by weight, and most preferably 80-98% by weight, based on a total amount of all monomers. Other monomers with functional groups may include for example crosslinkable acrylic monomers, such as (meth)acrylic acid, 2-hydroxyethyl (meth)acrylate, 4-hydroxybutyl (meth)acrylate, epoxypropyl (meth)acrylate and the like. Interaction with the ionic compound can be better controlled and the pressure-sensitive adhesive with better adhesion performance can be obtained by using monomers based on one or more types of alky (meth)acrylate having the alkyl group having 1 to 14 carbon atoms.

The type of polymerization for obtaining (meth)acryl polymer is not limited to a particular one. Solution polymerization, bulk polymerization, suspension polymerization, emulsion polymerization and photo-polymerization are preferred, and solution polymerization is more preferred. The polymerization may be initiated by an initiator. The initiators usable may include azo type initiators, such as azobisisobutyronitrile and azobiscyclohexanenitrile, and peroxides, such as benzoyl peroxide and acetyl peroxide. These initiators may be used in combination. The polymerization may be carried out at 50 to 100° C.

The weight average molecular weight of the aforesaid (meth) acryl-based polymer used in the present invention is preferably 100,000 to 5,000,000, more preferably 200,000 to 4,000,000, and most preferably 300,000 to 3,000,000. If it is less than 100,000, a residual paste (adhesive residue) tends to form due to reduced cohesion force of the pressure-sensitive adhesive composition. If it is greater than 5,000,000, the polymer tends to have a reduced flowability, the polarizing sheet tends to have an insufficient wettability, and peeling tends to occur between the polarizing sheet and the pressure-sensitive adhesive composition layer of the surface protection film. The weight average molecular weight is determined by gel permeation chromatography (GPC).

In addition, the aforesaid (meth)acryl-based polymer generally used has a glass transition point (Tg) not greater than 25° C., and it is preferably −90° C. to 0° C., and more preferably −80° C. to −10° C. If it is greater than 0° C., the polymer tends to have a reduced flowability, the polarizing sheet tends to have an insufficient wettability, and peeling tends to occur between the polarizing sheet and the pressure-sensitive adhesive composition layer of the surface protection film. Herein, the Tg of the (meth) acryl-based polymer may be adjusted to fall within the aforesaid range by properly modifying the monomers and the component ratios of the composition.

A specific example of the (meth)acryl-based polymer is a product from copolymerization of butyl acrylate (BA), acrylic acid (AA) and 2-hydroxyethyl methacrylate (HEMA) as comonomers, each used in an amount of 80 to 99% by weigh for BA, 0.1 to 15% by weight for AA, and 0.1 to 5% by weight for HEMA, based on the copolymerized products as 100% by weight.

In the pressure-sensitive adhesive composition of the present invention, the (meth) acryl-based polymer can be properly crosslinked for obtaining better heat-resistant pressure-sensitive adhesive sheets. Conventional crosslinking agents may be used for the crosslinking reaction. For example, a compound, such as isocyanate compound, epoxy compound, melamine-based resin or azacyclopropane compound, having a group reactive to carboxyl or hydroxyl group as crosslinking site in the (meth)acryl-based polymer can be added to the reaction. Among these, isocyanate compound and epoxy compound are preferred for obtaining proper cohesion force. These compounds can be used alone or in combination of two or more thereof.

The method of forming a pressure-sensitive adhesive layer on a film is not particularly limited. For example, it may be directly coated on a carrier film using a bar coater or a doctor blade, etc. As one of examples, the pressure-sensitive adhesive layer may be formed on a carrier layer through coating the carrier layer with the aforesaid pressure-sensitive adhesive composition and then drying it to remove the solvent.

The pressure-sensitive adhesive sheets of the present invention may be in a sheet or tape shape formed by coating one or both sides of various carrier layers with the aforesaid pressure-sensitive adhesive layer. The carrier layers may include a plastic film, such as a polyester film, or a porous material such as paper and unwoven fiber, with a thickness of 3 to 100 μm, and preferably 5 to 50 μm. Particularly, if a surface protection film is desired, a plastic substrate is preferably used as the carrier layer.

Some certain examples are described as follows to explain the present invention; however, the scope of the present invention is not limited to these examples.

### Test Methods

Determination of Glass Transition Point:

Glass transition point was determined by a differential scanning calorimeter (model: □100, made by TA instruments company, Taiwan) at a scanning rate of 10° C./min.

Tensile Test:

The pressure-sensitive adhesive sheet was cut into a strip-shaped specimen with a width of 2.5 cm. After the releasing film was removed, the specimen was placed and stuck on SUS316 standard stainless steel plate as being rolled down by a 2-kilogram rubber roller. The specimen was then tested for peeling force (180 degrees) at a drawing rate of 30 mm/min by a tensile tester (Model: Dachang, fabricated by Cometech Testing Machines Co., Ltd, Taiwan).

Holding Power:

The pressure-sensitive adhesive sheet was cut into a strip-shaped specimen with a width of 2.5 cm. After the releasing film was removed, the specimen was placed and stuck on SUS316 standard stainless steel plate through being pressed by a 2-kilogram rubber roller. The specimen was placed in an oven at 70° C. for 20 minutes and then placed in a holding power tester (Model: ChemInstruments HT-8, fabricated by ChemInstruments, Inc, Ohio), and a 1-kilogram weight was hung from the specimen. The time for the specimen to pull away from the stainless steel plate and the moving distance were determined after 40 minutes.

Surface Antistatic Test:

The surface antistatic test was performed by a device (Model: Hiresta-Up Mcp-HT450, commercially obtained from Mitsubishi Chemical Corporation, Japan). The device applied an external voltage of 10 to 1,000 volts on two concentric disc electrodes of the specimen and the surface resistance was read in Ω/□.

Molecular Weight Determination:

Molecular weight was determined by GPC device (600 Controller, manufactured by Waters International co.) under conditions: sample concentration: 0.2% by weight (THF solution); sample injection amount: 200 μm; eluent: THF; flow rate: 1.0 ml/min; column temperature: 40° C.; column: Shodex KF803 (column 1)+Shodex KF804 (column 2)+Shodex KF805 (column 3)+Shodex KF806 (column 4) manufactured by Waters International co.); and detector: refraction index detector. Calculation of molecular weight was based on a calibration line obtained using polystyrene standard.

Structural Analysis of the Ionic Compound:

The structural analysis of the ionic compound was performed by FT-IR determination and characterization.

FT-IR Test:

FT-IR was tested using an FT-IR spectrometer (Model: Spectrum One, manufactured by Perkin Elmer Co., USA) under following conditions: attenuated total reflectance (ATR) method detector, deuterated triglycine sulfate (DTGS) resolution: 4.0 cm⁻¹, and accumulation times: 32.

### Example

### Preparation Example Preparation of Methacrylate Polymer (A)

A four-necked flask equipped with a stirrer, thermometer, a nitrogen inlet and a condenser was charged with 189.4 parts by weight of butyl acrylate, 10 parts by weight of acrylic acid, 0.6 parts by weight of hydroxyethyl methacrylate, 0.16 parts by weight of 2,2′-azobisisobutyronitrile as a polymerization initiator and 466 parts by weight of ethyl acetate as a solvent. After nitrogen gas was introduced under mild stirring for about 30 minutes, the polymerization was carried out for about 8 hours, and the temperature of the liquid in the flask was maintained at about 60° C. during the polymerization. After it was cooled down to room temperature, 334 parts by weight of ethyl acetate was added into the flask for dilution, obtaining acrylate-based polymer (A) with a solid content of 20%. The polymer (A) has a glass transition point of −44° C. determined by differential scanning calorimetry and a weight average molecular weigh of 1,280,000.

### Synthesis Example Synthesis of the Ionic Compound

### Synthesis Example 1 Synthesis of the Ionic Compound (a)

45 parts by weight of 2-(acryloyloxy)ethyl trimethyl ammonium chloride aqueous solution in a concentration of 80% by weight was loaded in a stirring tank and was diluted with 45 parts by weight of pure water, followed by adding 10 parts by weight of acetone and 0.01 parts by weight of p-hydroxyl anisole. The temperature was controlled at from 4 to 8° C. 60 parts by weight of lithium bis(trifluoromethane-sulfonyl)imide dissolved in 60 parts by weight of pure water was dropped slowly into the stirring tank, and acetone was added in the same time for keeping the solution clear. Thereafter, the stirring was continued for about 3 hours. The acetone was evacuated by rotary concentration at room temperature. Then, extraction was performed twice by adding ethyl acetate and the organic phase was collected. The organic phase was added with a little amount of acetone and dewatered by anhydrous magnesium sulfate. After the magnesium sulfate was filtered off, the solvent was removed by rotary concentration to obtain a final product as a transparent liquid characterized by FT-IR spectroscopy to be 2-(acryloyloxy)ethyl trimethyl ammonium bis(trifluoromethane-sulfonyl) imide and noted as the ionic compound (a).

### Synthesis Example 2 Synthesis of the Ionic Compound (b)

### A magnetic Stirrer, 100 Parts by Weight of Ionic Compound (a),

400 parts by weight of ethyl acetate as a solvent, and 2 parts by weight of 2,2′-azobisisobutyronitrile as a polymerization initiator were loaded in a two-necked flask equipped with a nitrogen inlet and a condenser. Nitrogen was introduced into the flask for 30 minutes under mild stirring and the flask was heated to reach a temperature of 70° C. to carry out the polymerization for about 6 hours and then cooled down to room temperature, obtaining a light yellow transparent liquid as a solution of the ionic compound (b) with a solid content of 20%. The ionic compound (b) is an oligomer of the ionic compound (a) and has a glass transition point at 33° C. as determined by a differential scanning calorimeter.

### Synthesis Example 3 Synthesis of the Ionic Compound (c)

### A Magnetic stirrer, 50 Parts by Weight of Ionic Compound (a),

50 parts by weight of butyl acrylate, 400 parts by weight of ethyl methyl ketone as a solvent, and 2 parts by weight of 2,2′-azobisisobutyronitrile as a polymerization initiator were loaded in a two-necked flask equipped with a nitrogen inlet and a condenser. Nitrogen was introduced into the flask for 30 minutes under mild stirring and the flask was heated to reach a temperature of 80° C. to carry out the polymerization for about 6 hours and then cooled down to room temperature, obtaining a solution of the ionic compound (c) with a solid content of 20%. The ionic compound (c) is a copolymer of the ionic compound (a) and butyl acrylate.

### Example 1

### Preparation of the Pressure-Sensitive Adhesive Composition According to the Present Invention

0.1 parts by weight of the ionic compound (a) obtained from Synthesis Example 1 as described above was added to 100 parts by weight of 20% by weight of the acrylic polymer (A) solution obtained from Preparation Example as described above. 0.4 parts by weight of polyisocyanate adduct (i.e. toluene diisocyanate-trihydroxymethyl propane adduct, made by Nippon Polyurethane Industry Co., Ltd. (NPU)), 0.8 parts by weight of aluminum acetylacetonate, and 0.04 parts by weight of 3-glycidoxypropyltrimethoxysilane (Product code: Z-6040, made by Dow Corning Co., USA) were also added to serve as cross-linking agents and additives. The mixture was stirred at room temperature for about 5 minutes, obtaining an acrylic pressure-sensitive adhesive solution.

The preparation of the pressure-sensitive adhesive sheet

The acrylic pressure-sensitive adhesive solution obtained above was applied on a poly(ethylene terephthalate) (PET) film and heated at 90° C. for 3 minutes to form a pressure-sensitive adhesive layer with a thickness of 25 μm. Thereafter, a releasing side of a PET releasing film was pressed onto the other side of the pressure-sensitive adhesive layer, thereby forming a pressure-sensitive adhesive sheet. It was baked in an oven at 90° C. for 1 hour, resulting in a pressure-sensitive adhesive sheet which was tested for tensile strength, holding power, and surface antistatic properties.

### Example 2

### Preparation of the Pressure-Sensitive Adhesive Composition According to the Present Invention

An acrylic pressure-sensitive adhesive solution was prepared in a same way as that in Example 1, except that 1 part by weight, instead of 0.1 parts by weight, of the ionic compound (a) was added.

The preparation of the pressure-sensitive adhesive sheet

It is the same as that in Example 1.

### Example 3

### Preparation of the Pressure-Sensitive Adhesive Composition According to the Present Invention

An acrylic pressure-sensitive adhesive solution was prepared in a same way as that in Example 1, except that the ionic compound (b) (oligomer) solution with a solid content of 0.1 parts by weight obtained from Synthesis Example 2, instead of the ionic compound (a) was added.

The preparation of the pressure-sensitive adhesive sheet

It is the same as that in Example 1.

### Example 4

### Preparation of the Pressure-Sensitive Adhesive Composition According to the Present Invention

An acrylic pressure-sensitive adhesive solution was prepared in a same way as that in Example 3, except that the ionic compound (b) (oligomer) solution with a solid content of 1 part by weight, instead of 0.1 parts by weight, obtained from Synthesis Example 2 was added.

The preparation of the pressure-sensitive adhesive sheet

It is the same as that in Example 1.

### Example 5

### Preparation of the Pressure-Sensitive Adhesive Composition According to the Present Invention

An acrylic pressure-sensitive adhesive solution was prepared in a same way as that in Example 1, except that the ionic compound (c) (copolymer) solution with a solid content of 0.1 parts by weight obtained from Synthesis Example 3, instead of the ionic compound (a) was added.

The preparation of the pressure-sensitive adhesive sheet

It is the same as that in Example 1.

### Example 6

### Preparation of the Pressure-Sensitive Adhesive Composition According to the Present Invention

An acrylic pressure-sensitive adhesive solution was prepared in a same way as that in Example 5, except that the ionic compound (c) (copolymer) solution with a solid content of 1 part by weight, instead of 0.1 parts by weight, obtained from Synthesis Example 3 was added.

The preparation of the pressure-sensitive adhesive sheet

It is the same as that in Example 1.

### Comparative Example 1

### Preparation of the Pressure-Sensitive Adhesive Composition

0.4 parts by weight of polyisocyanate adduct as that in Example 1, 0.8 parts by weight of aluminum acetylacetonate, and 0.04 parts by weight of silane coupling agent (Product code: Z-6040) serving as cross-linking agents and additives were added to 100 parts by weight of the acrylic polymer (A) solution in a concentration of 20% by weight, obtained from Preparation Example described above, mixed at room temperature, and stirred for about 5 minutes, obtaining acrylic pressure-sensitive adhesive solution.

The preparation of the pressure-sensitive adhesive sheet

It is the same as that in Example 1.

### Comparative Example 2

### Preparation of the Pressure-Sensitive Adhesive Composition

An acrylic pressure-sensitive adhesive solution was prepared in a same way as that in Example 1, except that 1 part by weight of ionic liquid 1-propyl-3-methylimidazolium bis(trifluoromethane-sulfonyl)imide, instead of 0.1 parts by weight of the ionic compound (a), was added.

The preparation of the pressure-sensitive adhesive sheet

It is the same as that in Example 1.

Test

The resistance, tensile strength and holding power of the acrylic pressure-sensitive adhesive sheets from Examples 1-6 and Comparative Examples 1 and 2 were determined and the results are shown in Table 1. In view of Table 1, the resistance value decreases as the amount of the ionic compound (a) increases. The similar situation is also applicable to the ionic compound (b) (oligomer) and the ionic compound (c) (copolymer) that the resistance value is reduced as the amount of addition is increased. The resistance value is reduced from 10¹⁴Ω/□ to 10¹¹Ω/□ by adding just 0.1 parts by weight (based on the solid content) of the ionic compound, indicating that the pressure-sensitive adhesive sheets have excellent antistatic properties. For the tensile strength test, the tensile strength value is not changed significantly as the amount of the ionic compound (a) increases, and the tensile strength value increases slightly as the ionic compound (b) or (c) is added. The holding power is 0 mm for all. In comparison with the significant reduction of the tensile strength with the addition of the ionic liquid in Comparative Example 2, it is indicated that the pressure-sensitive adhesive sheets formed from the pressure-sensitive adhesive compositions according to the present invention have excellent adhesion.

TABLE 1
The resistance, tensile strength and holding power
of the acrylic pressure-sensitive adhesive sheet

| | PSA | Tensile | Holding | |
|---|---|---|---|---|
| | resistance | strength | power | |
| | (Ω/□) | (g/25 mm) | (mm) | Appearance |
| Ex. 1 | 1.33 × 10¹¹ | 653 | 0 | Clear |
| Ex. 2 | 3.49 × 10¹⁰ | 656 | 0 | Clear |
| Ex. 3 | 3.48 × 10¹¹ | 696 | 0 | Clear |
| Ex. 4 | 1.20 × 10¹¹ | 774 | 0 | Clear |
| Ex. 5 | 4.68 × 10¹¹ | 660 | 0 | Clear |
| Ex. 6 | 1.79 × 10¹¹ | 711 | 0 | Clear |
| Comp. Ex. 1 | >10¹⁴ | 663 | 0 | Clear |
| Comp. Ex. 2 | 2.83 × 10⁹ | 431 | 0 | Clear |

Those skilled in the art will readily observe that numerous modifications and alterations of the device and method may be made while retaining the teachings of the invention.

What is claimed is:

1. An ionic compound represented by the following chemical formula (I): 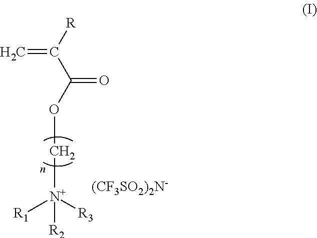
wherein, R is a hydrogen atom or a methyl group;
R₁, R₂, and R₃ are each independently an alkyl group having 1 to 6 carbon atoms; and
n is an integer of 1 to 16.

2. An oligomer comprising a product produced from oligomerization of an ionic compound as recited in claim 1 as a monomer.

3. A copolymer comprising a product produced from copolymerization of comonomers including an ionic compound represented by the following chemical formula (I): 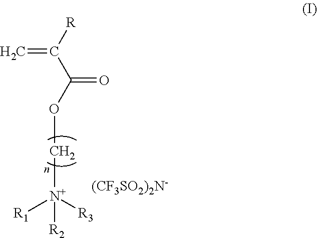
wherein, R is a hydrogen atom or a methyl group,
R₁, R₂, and R₃ are each independently an alkyl group having 1 to 6 carbon atoms, and
n is an inter of 1 to 16; and
at least an acryloyl-reactive comonomer.

4. A pressure-sensitive adhesive composition, comprising:
a first component selected from the group consisting of an ionic compound represented by the following chemical formula (I): 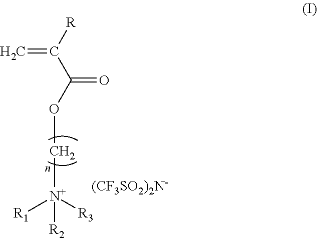
wherein, R is a hydrogen atom or a methyl group,
R₁, R₂, and R₃ are each independently an alkyl group having 1 to 6 carbon atoms, and
n is an inter of 1 to 16,
an oligomer of the ionic compound represented by the chemical formula (I), a copolymer of the ionic compound represented by the chemical formula (I) as a comonomer, and a combination thereof; and
a second component comprising a pressure-sensitive adhesive polymer.

5. The pressure-sensitive adhesive composition of claim 4, wherein the first component is present in an amount of from 0.01 to 20% by weight as calculated by the ionic compound, based on the total weight of the first component and the second component as 100% by weight.

6. The pressure-sensitive adhesive composition of claim 4, further comprising a solvent.

7. The pressure-sensitive adhesive composition of claim 4, wherein the pressure-sensitive adhesive polymer comprises a (meth) acrylic-based polymer having a weight average molecular weight of from 100,000 to 5,000,000.

8. The pressure-sensitive adhesive composition of claim 7, wherein the (meth)acrylic-based polymer comprises a product produced from copolymerization of butyl acrylate, acrylic acid and 2-hydroxyethyl methacrylate as comonomers.

9. The pressure-sensitive adhesive composition of claim 8, wherein butyl acrylate, acrylic acid and 2-hydroxyethyl methacrylate are present as comonomers in amounts of from 80% to 99% by weight, from 0.1% to 15% by weight, and from 0.1% to 5% by weight, respectively, based on the amount of the (meth)acrylic-based polymer as 100% by weight.
